(19) United States
(12) Patent Application Publication
(10) Pub. No.: US 2004/0151787 A1
(43) Pub. Date: Aug. 5, 2004

(54) Water soluble compositions derived from plant material and preparation thereof

(76) Inventors: Ronald W. Pero, Sandgate, VT (US); Lee Anthony Worth, Upper Montclair, NJ (US)

Correspondence Address: Patrick H. Higgins, Mathews, Collins, Shepherd & McKay, 100 Thanet Circle, Suite 306, Princeton, NJ, 08540, US

(21) Appl. No.: 10/718,165

(22) Filed: Nov. 20, 2003

Related U.S. Application Data
(60) Provisional application No. 60/428,090, filed on Nov. 21, 2002.

(51) Int. Cl. A61K 35/78

(52) U.S. Cl. 424/725

(57) ABSTRACT

A process for producing a composition of water-soluble phytomedicinal compounds is provided comprising combining plant material with water, in a ratio of plant material to water within a range of about 1:5 to about 1:50, at a temperature between about 75° C. and about 100° C. for a period of time to solubilize a substantial portion of thermal aqueous extractable phytocompounds present in the plant material, to produce a first extract; and removing substantially all entities having a molecular weight greater than about 10 kd from the extract to produce a composition of water-soluble phytomedicinal compounds. Compositions of water-soluble phytomedicinal compounds are provided that exhibit improved efficacy and reduced toxicity.

Priority is indicated herein from U.S. Provisional Application Ser. No. 60/428,090, filed Nov. 21, 2002.

### FIELD OF THE INVENTION

The present invention is directed toward a process for producing compositions of water-soluble phytomedicinal compounds, substantially devoid of molecular entities larger than about 10 kd, that exhibit enhanced therapeutic efficacy and reduced toxicity.

### BACKGROUND OF THE INVENTION

The most common use of phytomedicinal extracts, e.g., nutraceuticals and medicinal botanicals, is to treat chronic back pain, headache, depression, anxiety, fatigue, obesity, arthritis, insomnia, digestive problems, cardiovascular and cancer prevention, and aging. Conover, E. A., Clinical obstetrics and gynecology 45(1): 89-98, 2002; Williams, J. E., Alternative medicine review 6(6): 567-579, 2001; Block, J. B. and Evans, S., J. Am. Nutra. Asso. 3(3): 6-16, 2000. Particularly, human conditions where conventional pharmaceutical treatments do not work satisfactorily or have undesirable side-effects generally motivate nutritional approaches. Common justification for nutraceutical supplements include the well-known assertion that most foods available today lack nutritional quality as a result of changes in farming methods, choices of crops, harvesting fruits and vegetables before they are ripe, improper storage during transportation, processing, inadequate preparation by consumers, and contamination with herbicides, insecticides and fungicides. Whitman, M., Clin. J. Oncol. Nursing 5(5): 190-193, 2001. Hence, there is a great need for nutraceuticals to the general population to provide appropriate nutritional support.

Unambiguous evidence now exists that oxidative stress (i.e., the presence of highly reactive oxygen free radicals that damage DNA, RNA, and proteins) mediates the most serious human illnesses. Cross, C. E., Halliwell, B., Borish, E. T., Pryor, W. A., et al Ann. Intern. Med. 107: 526-545, 1987; Houston, M. C., Strupp, J. A., J. Amer. Nutra. Asso. 3(3): 1-5, 2000). Accordingly, antioxidant therapies are the hallmark of nutraceutical development because exogenous antioxidants such as carotenoids, flavonoids, vitamin C, Vitamin E, selenium, inter alia, have profound effects on human disease. The source of these antioxidants to human physiology is diet. Consequently, there is a need to optimize the consumption of dietary antioxidants as a protection against disease.

Uncaria water extracts have been reportedly produced by hot water extraction and filtered to produce a highly biologically active extract called C-Med-100 to enhance DNA repair and immune function, and inhibit tumor growth and inflammation. See, U.S. Pat. Nos. 6,238,675, 6,039,949 and 6,361,805.

A significant need, however, exists for the development of dietary supplements which provide bioavailable antioxidants from a wide variety of traditional herbs, for example, and which eliminate or minimize side effects of otherwise detrimental components of plant extracts.

### SUMMARY OF THE INVENTION

The present invention is directed to a process for producing a composition of water-soluble phytomedicinal compounds comprising combining plant material with water, in a ratio of plant material to water within a range of about 1:5 to about 1:50, at a temperature between about 75° C. and about 100° C. for a period of time to solubilize a substantial portion of thermal aqueous extractable phytocompounds present in the plant material, to produce a first extract; and removing substantially all entities having a molecular weight greater than about 10 kd from the extract to produce a composition of water-soluble phytomedicinal compounds.

In addition, the current invention is directed to processes wherein the resulting composition is substantially devoid of water-insoluble compounds.

The invention is further directed to compositions produced by processes described herein.

### BRIEF DESCRIPTION OF THE FIGURES

FIG. 1 displays several comparisons of in vitro data for anti-proliferation against HL60 wt (human leukemic cells wild type) using Garcinia extracts prepared by means of the process of the present invention.

FIG. 2 shows a comparison of the HL60 antiproliferation effects of two commercially available preparations of Garcinia extracts compared with Garcinia extract prepared by methods of the present invention.

### DETAILED DESCRIPTION OF THE INVENTION

Unless defined otherwise, all technical and scientific terms used herein have the same meaning as is commonly understood by one of skill in the art to which this invention belongs. All publications and patents referred to herein are incorporated by reference.

The present invention is drawn toward aqueous phytomedicinal extracts of plant species, which species possess valuable phytochemical and/or otherwise efficacious health properties and methods of preparation thereof. Here, it is particularly disclosed that because charged molecules such as tannins and phenolics, for example, aggregate into high molecular weight conjugates, efficacy of phytomedicinal extracts otherwise produced are generally reduced and toxicity is increased, as a result of detrimental properties conferred by these high molecular weight entities. A method of combining hot aqueous extraction and a means of removing high molecular weight entities from the extract is demonstrated to substantially improve pharmacological properties of phytomedicinal extracts. Particularly described herein are compositions of water-soluble phytomedicinal compounds including metabolites and phytocompounds prepared by removing pigments, toxic conjugates and inhibitors of active ingredients. Large molecular weight entities that cause toxic side effects and/or function as inhibitor(s) of otherwise efficacious phytocompounds including metabolites are specifically removed to substantially eliminate all entities more than about 10,000 daltons in molecular weight. Particularly, resulting water-soluble compositions of the present invention are substantially devoid of molecular entities larger than about 10 kd (10,000 daltons Molecular Weight (MW)).

Accordingly, high MW factors such as pigment and aggregates of toxic elements (in many cases artifacts of prevalent methods of production) and entities, for example, created by conjugation of naturally occurring charged molecules such as tannins, phenols, metals, proteins, polysaccharides, amines, and/or organic acids, can be removed as described herein to produce compositions and substantially improve pharmacological properties of phytomedicinal extracts. Particularly, undesirable colors, for example, may be removed in processes described herein without affecting biological activity such as is the case of green tea extract, or by removing one or more natural occurring inhibitors of efficacy as is indicated with Larch tree and red wine water extracts, or by simply reducing toxic side effects without reducing biological activity as is indicated with pine bark extract (pycnogenol). See, Example I.

Ancient medicines derived from plants generally involve water extraction and heat; however, currently available commercial ‘herbal’ products derived from plants are either basically homogenized plant tissue or organic solvent extracts. Methods of the invention described herein are applicable to produce a wide spectrum of valuable phytomedicinal extracts as well as measurable improvements on a wide spectrum of different commercially available plant extracts. The present invention is applicable to essentially any plant tissue, particularly plant tissue known to possess medicinal properties or any plant extract including extracts already obtained by methods including organic solvent extraction. Fundamentally, methods of the present invention are provided wherein the plant material is extracted with water preferably heated to about 100° C. for at least about 1 hour (water temperatures over 100° C. can be obtained, a well-known physical phenomena, under increased atmospheric pressure). The water-soluble plant extract is then produced by removal of molecular entities larger than about 10 kd, including insoluble particulate materials, for example, by chromatography, filtration, dialysis, or centrifugation.

Processes for producing a composition of water-soluble phytomedicinal compounds of the present invention are particularly preferred which comprise combining plant tissue with water, in a ratio of plant tissue to water within a range of about 1:5 to about 1:50, at a temperature between about 75° C. and about 102° C. for a period of time to solubilize a substantial portion of thermal aqueous extractable phytocompounds present in the plant tissue, to produce a first extract; and removing substantially all entities having a molecular weight greater than about 10 kd from the extract to produce a composition of water-soluble phytomedicinal compounds. Although trace amounts of insoluble material(s) may remain, preferred compositions of the present invention are substantially devoid of water-insoluble entities.

The term plant material, as used herein, refers to whole plant, for example, or any particular structure, substructure, organ or tissue, including but not limited to, leaves, roots, bark, stems, flowers, seeds, and fruit. The plant material may be fresh or dried, whole or homogenized, for example, by grinding, crushing, chopping, or blending. The term plant material, as used herein, encompasses compositions including organic or aqueous solutions of plant materials and/or extract including wine and other commercially available materials described herein. Preferred plant material includes, but is not limited to, larch, pine bark, red wine, Garcinia, green tea, bilberry, black cohosh, cayene, chamomile, chaste tree, cranberry, echinacea, eleuthero, ephedra, evening primrose, feverfew, flax, garlic, ginger, ginkgo, ginseng, golenseal, hawthorn, horse chestnut, kava, licorice, milk thistle, peppermint, saw palmetto, saint john's wort, black tea and valerian.

Application of the current invention to currently available commerical nutraceutical products prepared by organic solvent extraction, for example, is particularly valuable because organic solvents extract components varying in water solublity ranging from poorly soluble to moderately soluble. By thermal aqueous extraction, as defined herein, the poor water-soluble portion is removed from the much more bioavailable water-soluble fraction. Moreover, the water insoluble ingredients are rich in highly aromatic compounds which are well-known to possess properties (e.g., oxidants, carcinogens) toxic to human physiology (similar in properties to the molecular entities greater that about 10 kd referred to herein). Accordingly, processes of the invention described herein significantly enhance the efficacy and decrease the toxicity of medicinal plant extracts over conventional organic solvent extraction.

The phrase “a period of time to solubilize a substantial portion of thermal aqueous extractable phytocompounds present in the plant material”, as used herein is a functional definition of the time that is required to solubilize a substantial portion, e.g., at least about 40% of the water-soluble components of a sample that would become water-extractable under the same conditions for an extended period of time (e.g., 48 hours).

Processes of the present invention are preferred wherein the ratio of plant tissue to water in the extraction is within a range of about 1:10 w/v to about 1:40 w/v, preferrably the ratio of plant tissue to water in the extraction is within a range of about 1:20 to about 1:40, —or about 1:25 w/v to about 1:35 w/v. The water temperature of the extraction to solubilize thermal aqueous extractable phytocompounds is generally preferred to be between about 75° C. and about 105° C., preferably, between about 85° C. and about 102° C., more preferrably, between about 90° C. and about 100° C. The incubation period during this water-extraction step is generally between about 0.5 hours and about 48 hours to produce a first extract. Various times of incubation may be used. The timing of the incubation is not a material aspect of the present invention, hence the functional definition, supra. Accordingly, the incubation period during the water-extraction step may also be between about 0.5 hours and about 24 hours—or—in another embodiment, between about 0.5 hours and about 12 hours—or—in another embodiment, between about 1 hour and about 6 hours.

The term first extract, as used herein, refers to the basic or crude aqueous phase of the extract; however, it is indeed contemplated that the first extract may also include all materials including debris and water-insoluble materials, i.e., before removing substantially all entities having a molecular weight greater than about 10 kd, for example, in a removing step which employs large-scale chromatography. In other words, although an aqueous phase separation is preferred at this stage of the process, it is not necessarily required to practice the invention described herein. The step of removing substantially all entities having a molecular weight greater than about 10 kd from the extract to produce a composition of water-soluble phytomedicinal compounds can be accomplished by methods known in the art such as chromatography (e.g., large-scale columns), filtration, dialysis and centrifugation. Process embodiments of the present invention further comprise the additional step of drying the composition to produce a powder, for example. Green tea is an example of preferred material for use in methods of the present invention to produce valuable therapeutic compositions substantially devoid of pigment. Garcinia, moreover, is a preferred example source of plant material to produce therapeutic compositions of the present invention.

Ultra-filtration, for example, is a well defined scientific method for isolating and purifying substances. The principle of ultra filtration is to pass a composition (e.g., molecules dissolved in water) through a semi-permeable membrane that will allow the separation and fractionation of molecules based on their size/molecular weight. Ultra filtration is a key step in producing plant extracts described herein which have reduced toxicity and increased efficacy compared to currently available compositions, for example, that do not exclude plant tissue originating molecular entities greater than about 10 Kd, based on size. A myriad of ultra filtration products are available, for example, form Millipore, Billerica, Mass., which have a 10 kd cut-off for use with the present invention. Examples of commercially available ultra-filtration systems that are satisfactory to complete this step of the process are: (1) Membrane ultra-filtration using Amicon YC cellulose acetate membranes (Millipore), Biomax and Amicon PM high flow polyether sulphone membranes (Millipore), Ultra Amico YM cellulose discs (Millipore), GEA membrane filtration systems, and Supelco membrane-based filtration (Sigma-Aldrich). (2) Gel ultra-filtration using Matrix cellufine cellulose (Millipore), Sephadex LH-20, G-10, G-15, G-25, G-50, G-75, or G-100 (American Bioscience) and Bio-Gel P polysaccharide gels (Biorad). Accordingly, other means to exclude molecular entities originating from plant tissue, greater than about 10 kd, based on size, including, but not limited to, chromatography, including but not limited to, gel-filtration, dialysis, and centrifugation, for example, are intended to be within the scope of the methods of the invention as defined herein. Accordingly, two approaches to carrying out molecular size separation/sieving, intended to be within the scope of the present invention include (1) passing water solutes through a semi-permeable membrane and (2) chromatography described as gel filtration or molecular-sieve/gel permeation chromatography. Winzor, D. J., J Biochem Biophys Methods 56(1-3): 15-52, 2003. Superdex and Sephacryl products, for example, are readily available from Amersham Biosciences, Princeton, N.J. Semi-permeable membrane filtration is accomplished by creating a membrane with pores that allow only molecules less than a certain size to pass. An example is dialysis through cellulose bags that permit only molecules<13,000 MW to pass. Plant-derived material is placed inside the bag and then the dialysis bag is placed in water containing almost no solutes. The <13,000 MW compounds pass through the dialysis membrane to create an aqueous solution outside the dialysis membrane, thus separating <13,000 MW components from the >13,000 MW components (that remain inside the dialysis bag (membrane)). Examples of semi-permeable membranes appropriate for dialysis or ultra filtration can be made with poly ether sulphone, nitrocellulose, cellulose acetate, and polyurethane. Commercially available sources include well-known suppliers, e.g., BioRad (CA), Milipore (MA), and Amersham (NJ). Ultra filtration by chromatography, e.g., gel filtration chromatography, employs the same principle of separation by molecular size, but instead of passing water solutes through a semi-permeable membrane, the water solution is passed through a column packed with a gel containing water insoluble particles with varying abilities to retard solutes depending on their molecular size. In other words, the gel is semi-permeable to molecules depending on their size, so that high MW components are retarded much more than small MW components, and as the water solution is passed through the column containing the gel, they are separated according to size. Examples of ingredients that are efficient for making gels used in molecular sieving are agarose, superdex, silica xergels, starch, cellulose or Millipore/Sephadex products. A commercial separation of natural products by gel exclusion is presented in Example 1.

Examples of extracts to be improved include, for example, Larch tree extract from Prothera; Pine bark extract (Pycnogenol) (for example, Horphag Research Limited), Red wine extract (for example, Nutrivine supplied by M. Moers, IHT Health Products), and Green tea extract (for example, manufactured by Wuxi Mingxin Tea Biological Products Co., Ltd, China). The ratio between high and low molecular components in water extracts of plants vary greatly from one plant preparation to another. When considering larch tree, pine bark, or green tea, for example, prepared as hot water extracts, the dialyzable portions in certain embodiments of the present invention represent 14.8%, 70.4% and 98% respectively of the total amount of solids remaining after drying.

Medicinal uses for compositions described in this patent application include treatment for weight loss, anti-aging, immune enhancement, DNA repair enhancement, anti-inflammation, cancer prevention, fatigue/anxiety, pain, allergy, cardiovascular disease, and skin (topical) protection/care. Accordingly, the current invention includes a method of administering an effective amount of a composition of the present invention to effect at least one physiological condition selected from the group consiting of weight loss, anti-aging, immune enhancement, DNA repair enhancement, anti-inflammation, cancer prevention and/or control, reduced fatigue/anxiety, reduced pain, amelioration of allergy conditions, reduce cardiovascular disease conditions, and enhanced skin (topical) conditions.

Commercially Available Plant Products and Their Preparation Into Phytomedicinal Extracts of the Present Invention by Example Methods

1. Uncaria tomentosa bark powder

Supplied by: Oscar Schuler Egg, Lima, Peru

2. Larch tree water extract (Larix occidentalis)

Carbohydrates=≧85% (Lot # 2-LA-00362-01)

Manufactured by: Larex, Inc., 4815 White Bear Parkway

White Bear Lake, Minn. 55110

Distributed by: Prothera, Inc., Pleasanton, Calif. 94566

3. Pine bark extract (Pycnogenol)

Manufactured by: Horphag Research Ltd (U.S. Pat. No. 4,698,360)

Supplied by: IHT Health Products, 225 Long Ave., Hillside, N.J.

4. Red wine extract (Nutrivine, Lot no 297, A. N. Howard 25/8/98, supplied by M-Moers, IHT Health Products)

5. Green tea extract (50% EGCG (Lot # EGCG50-20020226)

Manufactured by: Wuxi Mingxin Tea Biological Products

Co., Ltd, China. Supplied by: AF Nutraceutical Group, Inc, Morristown, N.J. 07960

Regardless of whether the starting plant material is whole, homogenized, crushed or pulverized or extract, including dried extract, the material is suitable for producing phytomedicinal extracts of the present invention according to methods described herein. An example procedure is wherein 5 grams of plant material (e.g., Uncaria bark or larch, pycnogenol nutrivine or green tea extract) is mixed with 167 ml distilled water, heated to about 100° C. (100° C. is preferred) until the volume is reduced to ⅓ the starting volume. The mixture is then centrifuged at 3000×g for 15 min to remove particulate matter. The supernate is labeled—the original water extract—. An aliquot, e.g, 50 to 200 ml, of this solution is dialyzed twice in 1 liter distilled water for 24 h at about 4° C. (or ultrafiltered) to produce (high MW>10,000, inside dialysis bag) a corresponding dialyzed or ultra-filtrated fraction (low MW<10,000). Portions of each of these 3 water extracts (i.e. original, MW>>10,000 and MW<10,000) were lypholyzed to dryness and used to evaluate their formulation by color quality and biological activity.

Evaluation of Color Quality and Yield

The 3 water extracts (i.e. original, MW>10,000 and MW<10,000) were characterized by visual comparison of the colors both as powders and in water solution (i.e., p/s). In addition, the weights of the dialyzed and non-dialyzed preparations could be determined so that the porton of solubilized particulates present in each could be estimated and calculated as a % or the “original water extract” (i.e. weight of dialyzed fraction+non-dialyzed fraction=100%).

Evaluation of Efficacy of the Novel Water extracts of Plants

When preparing plants for human consumption of natural occurring beneficial phytocompounds including metabolites, it is important to duplicate as much as possible historical practices, e.g., of ancient cultures, in the preparation. Methods described herein comprise thermal treatment (about 100° C.) during aqueous extraction. Because many well known medicinal plants contain biologically active charged molecules such as tannins, which in turn can conjugate to each other as well as other compounds, particularly after heating, the therapeutic window or efficacy of natural medicines may be greatly enhanced by separating large molecules that are often formed by heat from the naturally occurring non-conjugated smaller ones. The efficacy of natural phytochemicals and metabolites are synergized by the novel combinations (compositions substantially devoid of entities greater than about 10 kd) produced by means of methods of the present invention. Part of the advantage provided by compositions of the present invention is realized by the lack of conjugates formed by heat or left behind by extractions other than water (i.e., organic solvents including alcohols).

Ultra-filtration has been added to heat and water extraction as a way to enhance efficacy by substantially reducing high molecular weight conjugated entities.

Necrosis in Raji cells was used as an indication of lysomal-based toxicity of phytomedicinal extracts of the present invention as well as their general efficacy by guaging the effects of the compositions on tumor cell proliferation and the transcription factor, NF-KB, that controls the essential body processes of apoptosis and inflammation.

Death by Necrosis and General Toxicity Assay

Necrosis is a type of cell death induced by tissue damage and leads to inflammation. In contrast, apoptotic cell death is a normal mechanism whereby the body removes unwanted cells. Thus, the cell membrane of an apoptotic cell remains intact until it has been removed by phagocytic cells in tissues and thus the general toxic side effects which cause inflammation are avoided. Moreover, apoptotic cell death is of considerable interest in cancer therapy as well as in treatment of autoimmune conditions.

Evidence of therapeutic properties of phytomedicinal extracts of the present invention is demonstrated herein by induce apoptosis in tumor target cells (HL-60) and reducing toxic side effects (shown by reducing necrosis in Raji cells).

The methodology for the identification of necrotic cells was carried out using vital staining of membrane integrity and the analysis of stained cells (i.e., dead cells taking up the stain) (based on Fluorescence Activator Cell Sorting (FACS)). For this purpose, HL-60 human leukemic cells were exposed to 0-5 mg/ml dose range for each plant extract and harvested 1-2 days after incubation at 37° C. The cells then were prepared in HBSS and aliquots of 10⁶ cells were stained with 7AAD in FACS-buffer (HBSS supplemented with 0.1% NaN₃ and 3% FCS (Gibco BRL, Life Technologies, Paisley, GB)). The cells were analyzed by FACS Calibur flow cytometry using Cell Quest software (Becton Dickinson, San Jose, Calif.). Dead cells (% total) were calculated for each dose range and IC values determined.

Cell proliferation assay. The anti-proliferative capacity of the phytomedicinal extracts of the present invention were determined by colormetric MTT assay. Schweitzer, et al., Experimantal Hematology 21: 573-578, 1993. Briefly, 10 μl of serial duplicate dilutions of the novel water plant extracts were added to 190 μl of cells from HL-60 or Raji (0.05×10⁶ cells/ml) in 96-well, flat-bottomed plates (Corning, N.Y.) to give a final concentration of 0-5 mg/ml of the extracts. Plates were incubated for 72 hours at 37° C. and then pulsed with 20 μl MTT (5 mg/ml, Sigma) and incubated for an additional 3 hours at 37° C. Reduced MTT was measured spectrophotometrically with an automated plate reader at 540 nm after lysis of cells with 150 μl of dimethylsulfoxide and 25 μl 0.1 M glycine buffer (pH 10.5).

70Z/3NF-kB expression assay (ATCC No. TIB 158): To induce IgM expression 25 μg/ml LPS is added for 24 hours. The test is done in 24 well cell culture clusters and 200,000 cells per well are cultured with appropriate drug concentrations. The drug is diluted to two times the final concentration in 0.5 ml culture medium so that 0.4×10⁶ cells in 0.5 ml culture medium are added. The cells are cultivated for 24 hours. If IgM induction is wanted, LPS is added after 4 hours. For 500 ml complete medium used in these assays 50 ml serum, 5 ml Hepes, 10 ml Sodium pyruvate, 0.5 ml Mercaptoethanol, 0.5 ml Gentamycin, and RPMI to 500 ml. The sources for these ingredients are from Life Technology: cell culture clusters (Costar, C 3524), medium (RPMI 1640, 218075-091), Hepes IM (15630-056), Sodium pyruvate 100 mM (11360-039), 2-Mercaptoethanol 50 mM (31350-010), Gentamycin 50 mg/ml (15750-045), and from Sigma: serum (F-7524), and from Boule: DIFCO E. coli 055:B5 and LPS (3120-25-0) diluted to 5 mg/ml in Hanks balanced salt solution. The expression of NF-KB is evaluated by FACS analysis by estimating K-expression (anti K-antibody, Southern Biotechnology or Kebo in Sweden) after 24 hours in cells harvested and washed once with FACS buffer (Hank's BSS supplemented with 3% FCS and 5 ml IM Hepes) in a 96 well plate. Accordingly, 0.5×10⁶ cells were stained with 7AAD plus anti-K antibody or only 7AAD according to normal FACS procedures.

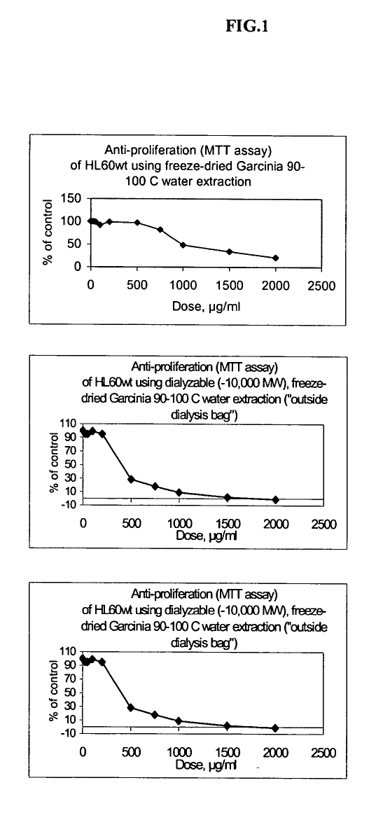

FIG. 1 shows several comparisons of Garcinia extracts prepared wherein water extraction of 12.15 gm Garcinia plant parts/500 ml distilled water was carried out at 90-100° C. for 8 hours, subjected to an ultra-filtration process which, in this case, was semi-permeable membrane bag dialysis to separate large molecular weight (MW) components (i.e. >13,000 MW) from small MW components (<13,000 MW), and then the 3 preparations were freeze-dried and bioassayed in vitro for anti-proliferation against HL60 wt (human leukemic cells wild type) using the MTT technology. Sheng, et al., Anticancer Res. 18:3363-3368, 1998. The IC₅₀ values for unfractionated Garcinia water extract prepared by this procedure, the >13,000 MW fraction and the (<13,000 MW) were 1000, 850 and 400 μg/ml, respectively, which in turn were calculated from the data presented in this Figure.

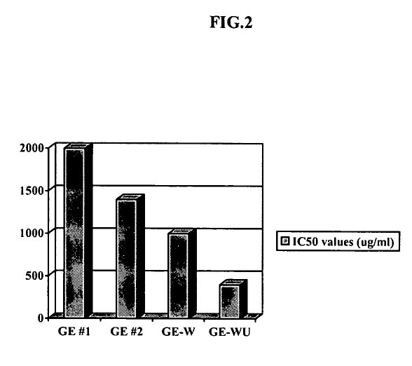

FIG. 2 shows a comparison of the HL60 antiproliferation effects of two commercially available preparations of Garcinia extracts with Garcinia extract prepared by methods of the present invention involving hot water extraction, ultra-filtration or both. GE #1=Garcinia extract precipitated with calcium hydroxide (Indfrag Limited, Eaton Town, N.J. GE #2=Garcinia extract precipitated with both calcium and potassium hydroxides (Super Citrimix, InterHealth). GE-W=Garcinia hot water extracted only (90-100° C.). GE-WU=Garcinia hot water extracted (90-100° C.) and ultra-filtrated (<13,000 MW components) were all bioassayed for toxicity (antiproliferation) to HL60 cells using a procedure as previously modified and described. Sheng et al., Anticancer Res. 18:3363-3368, 1998. IC₅₀ values were calculated from regression analyses of dose response similar to those found in FIG. 1.

The following examples are provided for the purpose of illustrating the value of the present invention in certain embodiments which employ water soluble plant extracts of Uncaria, larch tree, pine bark, red wine, and green tea; however, the scope of the invention is not limited to these examples but indeed encompasses the preparation of a wide variety of compositions of water-soluble phytomedicinal compounds, particularly from plant tissues having known medicinal value.

### EXAMPLES

### Example I

Preparation of phytomedicinal extracts according to the present invention decreases toxicity of the resulting composition and increases the efficacy of the pharmacological factors, e.g., compounds and metabolites, contained therein.

Herein provided are details of how to carry out processes of the invention on a large scale. This example is applicable to plant material to produce preparations substantially devoid of components>10,000 MW.

### Steps in the Procedure

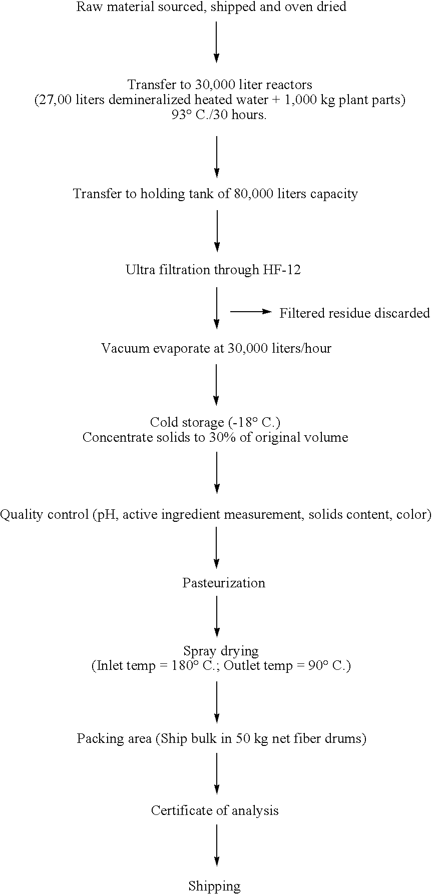

### Further Details of the Production Methodology

# 1 Extraction: The extraction was carried out in 3 steam-jacketed, stainless steel reactors each having a 27 cubic meter capacity. The milled plant parts (10 mm diameter) were extracted under 1.8 atmospheres of pressure with constant central agitation at 500 rpm in a ratio of 1 part of plant parts and 27 parts of water. The extraction was processed at 93° C. for 30 hours before being transported to the ultra-filtration section.

# 2 Filtration: The extract was ultra-filtered in 3 available units of Koch Romicon HF-12 systems, where each unit had 12 cartridges of Koch Romicon 5 inch diameter HF 66-60 having exclusion limits of 10,000 molecular weight. The ultra-filtration capacity was 2000 liters/hour and the filtrated material (i.e. <10,000 molecular weight) was stored in 30 cubic meter stainless steel storage tanks while waiting for final concentration under vacuum.

# 3 Concentration: Falling-film evaporators (APV, Inc., a Sao Paulo, Brazil Division of a British Co.) were used during the concentration processing stage having a water evaporation capacity of 30,000 liters/hour at 60-65° C. temperature. The concentrate was transferred to a 10 cubic meter stainless steel storage tanks for mixing and packing into 200 liter drums before storage at −18° C. Storage time was always less than 5 days before being spray dried.

# 4 Drying: The concentrated extract is stored in a cold storage room at −18° C. The product was pasteurized at 95° C. for 50-60 seconds before being spray dried at 180° C. at the inlet temperature and 90° C. at the outlet using a Niro F-10 Spray-Drier.

### EXAMPLE II

This example discloses the spectrum and variety of improvements in phytomedicinal extracts of the present invention by using the method of combining hot water extraction with ultra-filtration to produce extracts characterized as having about 100% water solubility and compounds of about 10,000d MW or below. Ultra-filtrated or dialyzed hot water extracts containing only solubilized low molecular weight components <10,000 MW of Uncaria, larch tree, pine bark, red wine and green tea all showed reduced color compared to the original extraction—or—the high molecular >10,000 MW fraction of the original extraction. Table 1, infra. These improvements in color were accompanied by either very minor changes in the dried mass weight of the original extracts such as was the case with green tea (e.g. being reduced to only 98% of the original), or a corresponding major dried mass difference that occurred upon dialysis of larch tree extract (i.e., only 14.8% of the original). High molecular weight components were removed from the dialyzed extracts. It is herein disclosed that all these dialyzed extracts showed substantial increased biological quality when evaluated for NF-kB inhibition, acute toxic side effects (necrosis) or inhibition of tumor cell (HL-60) growth (Table 1) as well as pigment (color) content. Taken together, these data clearly support the advantage of combining traditional medicine practices with heat and water extraction and removal of high molecular conjugates in order to produce formulations of natural products having increasing efficacy and reduced toxicity.

TABLE 1
Influence of ultra-filtration (dialysis) on quality of water extracts of plants.

| Water extracts | | Weight | Anti-NF-kB | HL-60 = IC₅₀ tox |
|---|---|---|---|---|
| of plants | Color | % of start | 70Z/E IC₅₀ | or Raji = IC_{necrosis} |
| Uncaria tomentosa extract | | | | |
| 1. Original(heat + water + particulates | dark brown | 100% | N.D. | HL-60 = 5/5 mg/ml |
| removed) | | | | |
| 2. Dialyzable portion | yellow tan | 54.8% | 0.5-1 mg/ml | HL-60 = 0.2-0.6 mg/ml |
| (compounds <10,000 MW) | | | | |
| 3. Non-dialyzable portion | brown | 45.2% | N.D. | HL-60 = 0.5-1.6 mg/ml |
| (compounds >10,000 MW) | | | | |
| Larch Tree Extract | | | | |
| 1. Original(heat + water + particulates | Cream white (p/s) | 100% | >5 mg/ml | Raji IC₂₅ = 1.25 mg/ml |
| removed) | | | | |
| 2. Dialyzable portion | Cream/beige (p/s) | 14.8% | 2.5 mg/ml | Raji IC₂₅ = 1.25 mg/ml |
| (compounds <10,000 MW) | | | | |
| 3. Non-dialyzable portion | Cream/white (p/s) | 85.2% | N.D | N.D. |
| (compounds >10,000 MW) | | | | |
| Pycnogenol extract | | | | |
| 1. Original(heat + water + particulates | Beige/beige (p/s) | 100% | 0.32 mg/ml | Raji IC₉₀ = <0.15 mg/ml |
| removed) | | | | |
| 2. Dialyzable portion | Beige/tan (p/s) | 70.4% | 0.32 mg/ml | Raji IC₉₀ = 1.25 mg/ml |
| (compounds <10,000 MW) | | | | |
| 3. Non-dialyzable portion | Beige/whiskey (p/s) | 29.6% | N.D. | N.D. |
| (compounds >10,000 MW) | | | | |
| Nutrivine extract | | | | |
| 1. Original(heat + water + particulates | Dark wine/wine (p/s) | N.D. | >2.5 mg/ml | Raji = <0.15 mg/ml |
| removed) | | | | |
| 2. Dialyzable portion | Light wine red | N.D. | 0.63 mg/ml | Raji = 1.26 mg/ml |
| (compounds <10,000 MW) | | | | |
| 3. Non-dialyzable portion | N.D. | N.D. | N.D. | N.D. |
| (compounds >10,000 MW) | | | | |
| Green tea extract (50% EGCG) | | | | |
| Lot # EGCG-20020226 | | | | |
| 1. Original(heat + water + particulates | Brown | 100% | N.D. | HL-60 = 12.5 μg/ml |
| removed) | | | | |
| 2. Dialyzable portion | White/cream | 98% | N.D. | HL-60 = 12.5 μg/ml |
| (compounds <10,000 MW) | | | | |
| 3. Non-dialyzable portion | Coco Brown | 2% | N.D. | HL-60 = 12.5 μg/ml |
| (compounds >10,000 MW) | | | | |

IC_{necrosis} = Inhibitory concentration dose of death by necorsis or general toxicity,
IC₅₀ tox = Inhibitory concentration dose at which 50% of proliferation occurs in HL-60 cells by apoptosis,
p/s = powder/soluble;
N.D. = not determined

### EXAMPLE III

The improved efficacy conferred to compositions of the present invention by eliminating molecular entities over 10,000 d is illustrated. Crude, unfractionated Garcinia hot water extract (6.8 gm) contains about 21%>13,000 MW components (1.4 gm) and 78%<13,000 MW components (6.8 gm). The ability to inhibit HL60 cell growth by the <13,000 MW fraction was enhanced 2½ times as evidenced by the change in IC₅₀ values from 1000 μg/ml to 400 μg/ml (1000/400=2.5) shown in FIG. 1. Accordingly, by separating out the >13,000 MW components of Garcinia water extract, the efficacy against HL60 cells was improved 2.5 fold.

### EXAMPLE IV

Significant improvement is illustrated by methods of the present invention over known procedures currently available for preparing Garcinia extracts. An important Garcinia active ingredient is alpha hydroxy citric acid. Jena, et al., Chemistry and biochemistry of hydroxcitric acid from Garcinia, J. Agric Food Chem. 50(1): 10-22, 2002. Alpha hydroxy citric acid is a simple low molecular organic acid easily precipitated with calcium/potassium hydroxide. In an effort to concentrate this active ingredient (indication for weight loss) in extracts, alpha hydroxy citric acid has generally been precipitated with either calcium hydroxide or potassium hydroxide or with both. Ohia, S. E., et al., Safety and mechanism of appetite suppression by a novel hydroxycitric acid extract (HCA-SX), Mol Cell Biochem 238(1-2): 89-103, 2002). These two preparations are identifed in FIG. 2 as GE #1 and GE #2. These preparations are compared directly with compositions of the present invention, i.e., prepared by the extraction procedure involving hot water extraction only (GE-W) or hot water extraction combined with ultra-filtration (GE-WU). The data recorded in FIG. 2 demonstrates the advantage of utilizing the procedure described herein for preparing plant extracts for the treatment of health disorders. Both GE-W and GE-WU were superior to either of the calcium or potassium hydroxide precedures in that, for this GE-W compositions are 1.4 to 2 times more efficacious and GE-WU is 3.5 to 5 more efficacious. Because the weight loss active ingredient present in Garcinia is low molecular weight and highly water soluble (alpha hydroxy citric acid), then both GE-W or GE-WU extracts are present along with other potential synergistic compounds which are not present when using the base precipitation procedures (e.g. Ca(OH)₂ and KOH). These data teach that, in general, by removing high molecular weight toxic elements and inhibitors of efficacy (i.e. >13,000 MW), methods described herein significantly improve pharmacological properties of phytomedicinal extract compositions produced thereby.

All publications and patents mentioned in the above specification are herein incorporated by reference. Various modifications and variations of the described compositions and methods of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described compositions and modes for carrying out the invention which are obvious to those skilled in the art or related fields are intended to be within the scope of the following claims.

What is claimed is:

1. A process for producing a composition of water-soluble phytomedicinal compounds comprising:
combining plant material with water, in a ratio of plant material to water within a range of about 1:5 to about 1:50, at a temperature between about 75° C. and about 102° C. for a period of time to solubilize a substantial portion of thermal aqueous extractable phytocompounds present in the plant material, to produce a first extract; and
removing substantially all entities having a molecular weight greater than about 10 kd from the extract to produce a composition of water-soluble phytomedicinal compounds.

2. The process according to claim 1 wherein the plant material is selected from the group consisting of leaves, bark, flowers, roots, stems, and fruit.

3. The process according to claim 1 wherein the composition is substantially devoid of water-insoluble compounds.

4. The process according to claim 1, wherein the ratio of plant material to water is within a range of about 1:10 to about 1:40, and the temperature is between about 75° C. and about 100° C., and the period of time is between about 0.5 hours and about 48 hours, which comprises the additional step of drying the composition.

5. The process according to claim 1 wherein the ratio of plant material to water is within a range of about 1:10 to about 1:40, and the temperature is between about 75° C. and about 100° C., and the period of time is between about 0.5 hours and about 24 hours.

6. The process according to claim 5 wherein the ratio of plant material to water is within a range of about 1:10 to about 1:40, and the temperature is between about 75° C. and about 100° C., and the period of time is between about 0.5 hours and about 12 hours.

7. The process according to claim 6 wherein the ratio of plant material to water is within a range of about 1:10 to about 1:40, and the temperature is between about 90° C. and about 100° C., and the period of time is between about 1 hour and about 6 hours.

8. The process according to claim 7 wherein the ratio of plant material to water is within a range of about 1:20 to about 1:40, and the temperature is between about 95° C. and about 1001° C., and the period of time is between about 1 hour and about 6 hours.

9. The process according to claim 8 wherein the ratio of plant material to water is within a range of about 1:25 to about 1:35, and the temperature is between about 95° C. and about 100° C., and the period of time is between about 1 hour and about 6 hours.

10. The process according to claim 1 wherein the plant material is homogenized.

11. The process according to claim 1 wherein the plant material is known to possess medicinal properties.

12. The process according to claim 1 wherein the step of removing substantially all entities having a molecular weight greater than about 10 kd from the extract is accomplished by means selected from the group consisting of ultra-filtration, chromatography, dialysis, and centrifugation.

13. The process according to claim 11 wherein the plant material is selected from the group consisting of larch tree, pine bark, red wine, Garcinia, and green tea.

14. The process according to claim 13 wherein the plant material is derived from green tea and the composition is substantially devoid of pigment.

15. A process for producing a composition of water-soluble phytomedicinal compounds comprising:
combining plant material with water, in a ratio of plant material to water within a range of about 1:5 to about 1:50, at a temperature between about 75° C. and about 102° C. for a period of time to solubilize a substantial portion of thermal aqueous extractable phytocompounds present in the plant material, to produce a first extract; and
removing substantially all entities having a molecular weight greater than about 13 kd from the extract to produce a composition of water-soluble phytomedicinal compounds.

16. The process according to claim 15 wherein the plant material is selected from the group consisting of larch tree, pine bark, red wine, Garcinia, and green tea.

17. The process according to claim 16 wherein the plant material is derived from Garcinia.

18. A composition of water-soluble phytomedicinal compounds produced by combining plant material with water, in a ratio of plant material to water within a range of about 1:5 to about 1:50, at a temperature between about 75° C. and about 102° C. for a period of time to solubilize a substantial portion of thermal aqueous extractable phytocompounds present in the plant material, to produce a first extract; and
removing substantially all entities having a molecular weight greater than about 10 kd from the extract to produce a composition of water-soluble phytomedicinal compounds.

19. A composition of water-soluble phytomedicinal compounds produced by the process of claim 18 wherein the plant material is selected from the group consisting of larch tree, pine bark, red wine, Garcinia, and green tea.

20. A composition of water-soluble phytomedicinal compounds produced by the process of claim 19 wherein the plant material is selected from the group consisting of Garcinia and green tea.

21. A method of administering an effective amount of a composition of water-soluble phytomedicinal compounds to effect at least one physiological condition selected from the group consiting of weight loss, anti-aging, immune enhancement, DNA repair enhancement, anti-inflammation, cancer prevention and/or control, enhance gastrointestinal digestion, reduced fatigue/anxiety, reduced pain (including headache), amelioration of allergy conditions, reduce cardiovascular disease conditions, and enhanced skin (topical) conditions, wherein said composition of water-soluble phytomedicinal compounds was prepared by a process comprising combining plant material with water, in a ratio of plant material to water within a range of about 1:5 to about 1:50, at a temperature between about 75° C. and about 102° C. for a period of time to solubilize a substantial portion of thermal aqueous extractable phytocompounds present in the plant material, to produce a first extract; and
removing substantially all entities having a molecular weight greater than about 1 Okd from the extract.
